(19) United States
(12) Patent Application Publication
(10) Pub. No.: US 2003/0054980 A1
(43) Pub. Date: Mar. 20, 2003

(54) Ceruloplasmin and uses thereof in neurodegenerative related conditions

(76) Inventors: Mircea Alexandru Mateescu, Ouest (CA); Joanne Paquin, Montreal (CA); Domynick Maltais, St-Isidore (CA)

Correspondence Address: MERCHANT & GOULD PC, P.O. BOX 2903, MINNEAPOLIS, MN, 55402-0903, US

(21) Appl. No.: 10/275,153

(22) PCT Filed: May 2, 2001
(86) PCT No.: PCT/CA01/00618

(30) Foreign Application Priority Data
May 3, 2000 (CA) 2,307,475

(51) Int. Cl. A61K 38/41; C12N 5/08

(52) U.S. Cl. 514/6; 435/368

(57) ABSTRACT

The present invention relates to the use of ceruloplasmin (and/or derivatives thereof) for treating neurodegenerative related conditions. More particularly, the present invention pertains to the use of ceruloplasmin in pharmaceutical neurotrophic compositions, to its use as a carrier for delivering therapeutic agent(s) to tissues of the nervous systems and to its use in methods for culturing neuronal cells in vitro. The present invention is useful for maintaining or stimulating the regeneration of neuronal cells, particularly for the treatment of injuries to the brain, the spinal cord and other central nervous system tissues.

### BACKGROUND OF THE INVENTION

1) Field of the Invention

The present invention relates to the use of ceruloplasmin (and/or derivatives thereof) for treating neurodegenerative related conditions encountered during development and ageing. More particularly, the present invention pertains to the use of ceruloplasmin in pharmaceutical neurotrophic compositions and in methods for culturing neuronal cells in vitro for promoting the tissular organization of these cells via their aggregation.

2) Description of the Prior Art

Nerve damage may occur as a consequence of inherited or congenital neurodiseases such as Rett's syndrome and epilepsy. It may further occur through physical injury, which causes the degeneration of the axonal processes and/or nerve cell bodies near the site of injury. Nerve damage may also occur because of temporary or permanent cessation of blood flow to parts of the nervous system, as in stroke or at birth. Nerve damage may also occur because of intentional or accidental exposure to neurotoxins, such as the cancer and AIDS chemotherapeutic agents cisplatinum and dideoxycytidine (ddC), respectively. Nerve damage may also occur because of chronic metabolic diseases, such as diabetes or renal dysfunction. Nerve damage may also occur because of neurodegenerative diseases such as Parkinson's disease, siderosis, Alzheimer's disease, and Amyotrophic Lateral Sclerosis (ALS), which result from the degeneration of specific neuronal populations.

A number of substances are known to influence neuronal development, including growth factors, components of the extracellular matrix, and cell adhesion and guidance molecules. Although a lot of efforts have been made to obtain factors and compositions for promoting the regeneration of neuronal cells, no ideal therapeutic agent exists yet. It would therefore be highly desirable to have such a therapeutic agent.

Ceruloplasmin (CP) is a multifunctional copper-glycoprotein of 132 kDa produced by hepatocytes and found in plasma (1.5-3 μM). This protein has important antioxidant and free radical scavenging properties towards reactive oxygen species (ROS) as well as a ferroxidase I activity. In particular, CP was shown as an important oxygen free radical (OFR) scavenger. Recent studies related to the alterations in the level of ceruloplasmin further support a dominant role of this protein, suggesting possible therapeutic applications. For example, international patent application No WO 98/25954 relates to the use of modified ceruloplasmin comprising a glycosylphosphatidylinositol moiety and its use for the treatment of toxic level of ferrous iron. Although the ROS scavenging capacities of CP have been shown in vitro, none of these studies has suggested the use of CP as neurotrophic factor neither have they shown that CP could act to stimulate the maturation, growth, tissular organization and/or regeneration of neuronal cells.

In view of the above, it is clear that there is a need for a pharmaceutical neurotrophic composition comprising ceruloplasmin (and/or derivatives thereof) for protecting neuronal cells, for promoting the tissular organization of neuronal cells and the regeneration of neuronal tissues. There is also a need for a composition which would achieve these needs by promoting neuronal cells aggregation.

The purpose of this invention is to fulfil these needs along with other needs that will be apparent to those skilled in the art upon reading the following specification.

### SUMMARY OF THE INVENTION

The present invention relates to the use of ceruloplasmin (and/or functional derivatives thereof) in neurotrophic compositions useful for treating neuropathologies, in particular neurodegenerative related conditions. More particularly, the present invention pertains to the use of ceruloplasmin for promoting the tissular organization of neuronal cells via their aggregation.

According to a first aspect, the invention provides a pharmaceutical neurotrophic composition which comprises ceruloplasmin and/or of a functional derivative of ceruloplasmin in an amount effective to protect neuronal cells and/or promote maturation, growth, tissular organization, regeneration and/or aggregation of neuronal cells; and a suitable pharmaceutical acceptable diluent or carrier.

According to another aspect of the invention, ceruloplasmin and/or its functional derivatives, are used as an active agent in the preparation of a medication for preventing or treating a neurodegenerative disease or for treating an injury to tissues of the central or the peripheral nervous system. The invention also provides methods for preventing or treating a neurodegenerative disease or for treating an injury to tissues of the nervous system, comprising the administration to a patient in need thereof of a therapeutically effective amount of ceruloplasmin or of a functional derivative of ceruloplasmin or the administration of a therapeutically effective amount of a neurotrophic composition as defined hereinabove.

According to another aspect of the invention, ceruloplasmin or its functional derivative is used as a transporter for delivering therapeutic agent(s) to specific tissues such as tissues of the nervous system. More particularly, the present invention provides methods for delivering therapeutic agent(s) to tissues of the nervous system comprising the use of ceruloplasmin or of a functional derivative thereof as a carrier for the therapeutic agent(s). These methods are particularly usefull for preventing or treating neurodegenerative diseases or for treating tissue injuries of the nervous system.

According to a preferred embodiment, the therapeutic agent(s) is coupled to ceruloplasmin or a functional derivative thereof by using coupling methods known in the art. The “conjugated” or “modified” ceruloplasmin so obtained is then administered, preferably via parental way, to a patient in need of the therapeutic agent(s).

According to a further aspect, the invention relates to a commercial package containing as an active pharmaceutical ingredient ceruloplasmin or a functional derivative of ceruloplasmin, together with instructions for the use thereof for protecting neuronal cells and/or for promoting maturation, growth, tissular organization, regeneration and/or aggregation of neuronal cells.

Neurodegenerative diseases which could be treated using the methods and package of the present invention include neuronal sequels or trauma caused by adverse developmental conditions that may be encountered during gestation or perinatally, Alzheimer's disease, stroke, sequels or damages or trauma to tissues of the nervous system (physical injury, temporary or permanent cessation of blood flow, exposure to neurotoxins, etc.) multiple sclerosis, Parkinson's disease, siderosis, HIV infection of the nervous system, AIDS dementia, amyotrophic lateral sclerosis, hereditary hemorrhage with amyloidosis-Dutch type, cerebral amyloid angiopathy, Down's syndrome, spongiform encephalopathy and Creutzfeld-Jakob disease.

According to a further aspect, the invention provides a method for protecting neuronal cells cultured in vitro and/or for promoting maturation, growth, tissular organization, regeneration and/or aggregation of neuronal cells cultured in vitro. The method comprises the step of providing to the in vitro cultured neuronal cells an effective amount of ceruloplasmin or of a functional derivative thereof.

An advantage of the present invention is that it provides effective means for maintaining or stimulating the regeneration of neuronal cells, and thereby, it permits the treatment of injuries to the brain, the spinal cord and other central nervous system tissues. Another advantage of the present invention is that it provides a carrier for delivering therapeutic agent(s) to specific tissues such as the brain. A further advantage of the invention is that it improves the efficiency of methods for culturing neuronal cells in vitro either as model system or graft material.

Other objects and advantages of the present invention will be apparent upon reading the following non-restrictive description of several preferred embodiments made with reference to the accompanying drawings.

### DESCRIPTION OF THE DRAWINGS

FIGS. 1A and 1B are pictures showing the morphology of newly differentiated P19 neurons cultured in absence (1A) or in presence (1B) of ceruloplasmin. P19 cells were induced to differentiate to neurons in the presence of retinoic acid (RA). On day 4 of differentiation, cells were trypsinized and plated (1500-1800 cells/mm²) on gelatin-coated tissue culture dishes and cultured in the absence (1A) or presence (1B) of 3.8 μM CP. Shown are phase-contrast photomicrographs of P19 neurons taken 24 h after plating.

FIGS. 2A, 2B and 2C are graphs showing a concentration-response curve of the pro-aggregative effect of native CP on P19 neurons. At day 4 of differentiation, dissociated P19 neurons were plated (1500-1800 cells/mm²) on gelatin-coated dishes and CP added 3 h later at indicated concentrations. Incubation was resumed for 24 h and the pro-aggregative effect of CP was assessed by three methods. FIG. 2A: Examination of culture morphology under microscope: 0, no restriction in cell spreading (morphology was as for cells in FIG. 1A); +, slight restriction in cell spreading; ++, moderate restriction in cell spreading; +++, high restriction in cell spreading with a tendency to form aggregates; ++++, intense cell aggregation (morphology was as for cells in FIG. 1B). FIG. 2B: Measurement of the portion of culture surfaces not occupied by cells. Three micrographs were taken from each condition and values reported as means±S.D. The results shown for one experiment are representative of three independent studies. FIG. 2C: Reduction of ALAMARBLUE™ dye by cells. The dye was added to the culture medium at a final concentration of 10% v/v and incubated with cells for approximately 5 h. Relative level of reduction was monitored by fluorescence. The values are reported as means±S.D. for triplicate determinations. The results are shown for one experiment and are representative of three independent studies. With all assays, a plateau value was reached at 0.38 μM and maintained until at least 3.8 μM.

FIG. 3 is a graph showing the influence of cell ageing on the response of P19 neurons to the aggregative effect of CP. Neurons derived from P19 cells were plated on gelatin-coated culture dishes at day 4 of the differentiation protocol. Different concentrations of native CP were added to the culture medium at 3, 24 or 48 h after plating. Neuronal cultures were left for 24 h with CP, and then photographed for analysis of unoccupied surfaces. The values are reported as mean±SEM for triplicate determinations.

FIG. 4 is a graph showing the aggregative effect of different forms of CP on P19 neurons. P19 neurons were obtained and treated as indicated in the legend of FIG. 2, except that deglycosylated CP and heat-denatured CP were also used. The extent of aggregation was scored as in FIG. 2A. Concentration of heat-denatured CP up to 7.6 μM failed to induce aggregation. Results are expressed as the average of at least three independent experiments.

FIGS. 5A and 5B are photomicrographs showing the effect of serine protease inhibitors on the neuroaggregative action of CP on P19 neurons. Neurons were obtained from the differentiation of P19 cells with RA and plated on gelatin-coated culture dishes at day 4 of differentiation. FIG. 5A: photomicrograph of neurons cultured 24 h with CP without inhibitors. FIG. 5B: photomicrograph of neurons cultured 24 h with CP+aprotinin+SBTI. Inhibitors (aprotinin and SBTI) were added to the culture medium 2 h after plating (6-7 TIU/mL each), followed 1 h later by native CP (3.8 μM). Complete inhibition of aggregation was observed when SBTI and aprotinin were tested individually, at the same concentration or even at half this concentration.

### DETAILED DESCRIPTION OF THE INVENTION

This application describes a novel neurotrophic factor. As used herein, “neurotrophic factor” or “neurotrophic composition” refers to any compound (or to any mixture of compounds) that promotes the maturation, growth, tissular organization, regeneration and aggregation of neuronal cells and/or protect these cells against a variety of different forms of damage. such as defective or incomplete neuronal development (e.g. Rett's syndrome, neuronal migration disorders as described by A. J. Copp and B. N. Harding, 1999, Epilepsy Res. 36, 133-141), acute nerve damage (e.g. stroke, trauma, epileptic episodes, secondary drug effects), chronic neurodegenerative status (e.g. sequels of various acute nerve damages, Alzheimer's disease, Parkinson's disease, Hallervoden-Spatz' disease, siderosis), and nerve damage caused by general metabolic pathologies (e.g. diabetes, renal dysfunction).

More particularly, the present invention describes the use of ceruloplasmin in a pharmaceutical neurotrophic composition and in a method for promoting the tissular organization of neuronal cells via their aggregation e.g. via the modulation of neuronal cells contacts. The neuronal cells that are most susceptible to benefit from the composition of the invention are newly differentiated neurons or highly developed neurons susceptible to undergo or to respond to a regenerative process.

As stated previously, ceruloplasmin (CP) is a 132 kDa multifunctional blue-copper plasma protein which is produced by hepatocytes and found in circulating plasma (1.5-3 μM). Its most known function is the copper transport. Ceruloplasmin also has important antioxidant and free radical scavenging properties as well as a ferroxidase I activity. Another important role has recently been postulated for this protein as a regulator of iron metabolism (David and Patel, 2000).

The ceruloplasmin and functional derivatives thereof according to the present invention are preferably substantially pure ceruloplasmin that has been purified from blood or produced by recombinant techniques. As generally understood and used herein, the term substantially pure refers to a ceruloplasmin preparation that is generally lacking in other cellular or blood components. Ceruloplasmin may advantageously be purified from blood using a one-step affinity chromatography method and an affinity column comprising aminoethyl-agarose or by any other protocol yielding a protein with comparable or ameliorated properties.

A “functional derivative”, as is generally understood and used herein, refers to a protein sequence that possesses a functional biological activity that is substantially similar to the biological activity of the whole protein sequence. A functional derivative of a protein may or may not contain post-translational modifications such as covalently linked carbohydrate, if such modification is not necessary for the performance of a specific function. The term “functional derivative” is intended to the “fragments”, “segments”, “variants”, “analogs” or “chemical derivatives” of a protein.

The terms “fragment” and “segment” as are generally understood and used herein, refer to a section of a protein, and are meant to refer to any portion of the amino acid sequence.

The term “variant” as is generally understood and used herein, refers to a protein that is substantially similar in structure and biological activity to either the protein or fragment thereof. Thus two proteins are considered variants if they possess a common activity and may substitute each other, even if the amino acid sequence, the secondary, tertiary, or quaternary structure of one of the proteins is not identical to that found in the other.

The term “analog” as is generally understood and used herein, refers to a protein that is substantially similar in function to ceruloplasmin.

As used herein, a protein is said to be a “chemical derivative” of another protein when it contains additional chemical moieties not normally part of the protein, said moieties being added by using techniques well known in the art. Such moieties may improve the protein solubility, absorption, bioavailability, biological half life, and the like. Any undesirable toxicity and side effects of the protein may be attenuated and even eliminated by using such moieties. For example, CP and CP fragments can be covalently coupled to biocompatible polymers (polyvinyl-alcohol, polyethylene-glycol, etc) in order to improve stability or to decrease antigenicity. They could also be coupled to proteins (or their chemical derivatives) known to pass the blood-brain barrier via transcytosis across vascular endothelial cells (e.g. covalent coupling to transferrin or to an antibody against transferrin receptors in order to be transported via endogenous transferrin transport systems). Association of both strategies (coupling to biocompatible polymers and to a ligand/agonist of cellular transport systems) may also be envisaged.

The amount of ceruloplasmin and/or functional derivatives thereof present in the neuroprotective composition of the present invention is a therapeutically effective amount. A therapeutically effective amount of ceruloplasmin is that amount of ceruloplasmin or derivative thereof necessary so that the protein act as a neurotrophic factor, and more particularly the amount necessary so that the protein promote the tissular organization of neuronal cells, increase the aggregation (association) of neuronal cells. The exact amount of ceruloplasmin and/or functional derivatives thereof to be used will vary according to factors such as the protein biological activity, the type of condition being treated as well as the other ingredients in the composition. Typically, the amount of ceruloplasmin should vary from about 0.001 μM to about 20 μM. In a preferred embodiment, ceruloplasmin is present in the composition of the neuroprotective extracellular medium in an amount from about 0.005 μM to about 10 μM, preferably from about 0.01 μM to about 5 μM. In the preferred embodiment, the neuroprotective composition comprises about 1 μM of highly active ceruloplasmin.

Further therapeutic agents can be added to the neurotrophic composition of the invention. For instance, the composition of the invention may also comprise therapeutic agents such as modulators of brain functions (neurotransmitters, neuropeptides, hormones, others trophic factors, or analogs of these substances that act by binding to brain receptors (e.g. DOPA in Parkinson's disease), modulators of brain cell adhesion/migration (proteases, protease inhibitors, chemorepulsive or chemoattractant substances, compounds of extracellular matrix, or analogs of these substances), compounds that help/assist the passage across the blood-brain barrier and neurotherapeutic chemical compounds (e.g. antioxidants to diminish or prevent damages caused by oxidative stress).

Further to the therapeutic agents, the pharmaceutical compositions of the invention may also contain metal chelators (proteinic or not), metal scavengers (proteinic or not), coating agents preserving agents, solubilizing agents, stabilizing agents, wetting agents, emulsifiers, sweeteners, colorants, odorants, salts, buffers, coating agents and/or antioxidants. For preparing such pharmaceutical compositions, methods well known in the art may be used.

The method of preparation of the neuroprotective composition of the invention consists simply in the mixing of purified ceruloplasmin and other component(s) in a buffered saline solution in order to get a homogenous physiological suspension. A suitable saline solution comprises sodium, potassium, magnesium and calcium ions at physiological concentrations, and has an osmotic pressure varying from 280 to 340 mOsmol, and a pH varying from 7.0 to 7.4. Preferably, the buffered saline solution is selected from the group consisting of modified Krebs-Henseleit buffer (KH) and phosphate buffer saline (PBS), both at pH 7.4. The homogenous suspension obtained can further be centrifuged and/or filtered to reduce its viscosity and/or eliminated non-soluble particles.

The neurotrophic composition of the invention could be suitable to treat and/or prevent neurodegenerative diseases or treat an injury to nervous system tissues. Neurodegenerative diseases which could be treated include Alzheimer's disease, stroke, trauma or damage of sequels to tissues of the nervous system, multiple sclerosis, Parkinson's disease, Hallervoden-Spatz' disease, siderosis, HIV infection of the nervous system, AIDS dementia, amyotrophic lateral sclerosis, hereditary hemorrhage with amyloidosis-Dutch type, cerebral amyloid angiopathy, Down's syndrome, spongiform encephalopathy, Creutzfeld-Jakob disease, neuronal disorders that affect development of the nervous system (e.g. agyria, heterotopia, cortical displasia), metabolic diseases susceptible to affect normal development of the nervous system such as diabetes and kidney insufficiency, and congenital neurodiseases such as Rett's syndrome, epilepsy, neuronal migration diseases, metabolic status or diseases affecting neuronal function/survival and nervous system damages caused by cessation of blood flow.

The neurotrophic composition could also be involved in the treatment of poisoning or diminution of side effects of drugs (such as chemotherapeutic and immunosuppressive drugs) to the brain and/or to neuronal cells.

The neurotrophic composition of the invention may be administered alone or as part of a more complex pharmaceutical composition according the desired use and route of administration. For instance, the neuroprotective composition of the invention could comprise a vector, such as a plasmid or a virus, comprising a DNA sequence coding for native ceruloplasmin, coding for a modified/fusion ceruloplasmin protein having an increased neurotrophic activity, an increased stability or an increased capacity of traversing the hematoencephalic barrier. Anyhow, for preparing such compositions, methods well known in the art may be used.

Using methods known in the art, ceruloplasmin or its functional derivative could also be coupled, via covalent linkage or tight non-covalent association, to one or more therapeutic agent(s) or vice versa. The “conjugated” or “modified” ceruloplasmin protein could then be administered to a patient in need thereof and thereby acts as a carrier for delivering therapeutic agent(s) to a known specific tissue such as the nervous system and more particularly to the brain, the spinal cord and the tissues of the peripheral nervous system. According to a preferred embodiment, the therapeutic agent is selected from the group consisting of neurotransmitters, neuropeptides, hormones, trophic factors, neurotherapeutic chemical compounds, modulators of cell adhesion/migration, modulators of axonal sprouting, modulators of axonal guidance, modulators of synaptic connections and analogs thereof capable of binding to tissues of the nervous system. For instance, conjugates of ceruloplasmin could be realized with transferrin or with an antibody against transferrin receptors which are large macromolecules, or with other molecules of similar type that are able to pass the blood-brain barrier. For other neuronal tissues in various organs, ceruloplasmin or its derivatives can be used as such, or only partly modified, even in the absence of transferrin or other conjugated molecules.

Ceruloplasmin and/or its derivatives may also be coupled to a biocompatible polymer (e.g. polyethylene glycol, polyvinyl alcohol) to reduce antigenicity when administered parenterally.

The neurotrophic composition of the invention and/or more complex pharmaceutical compositions comprising the same may be given via various routes of administration. For instance, the neurotrophic composition may be administered in the form of sterile injectable preparations, for example, as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intramuscularly or sub-cutaneously by injection or by infusion. Suitable dosages will vary, depending upon factors such as the amount of each of the components in the composition, the desired effect (fast or long term), the disease or disorder to be treated, the route of administration and the age and weight of the individual to be treated.

Other ways that can be considered are rectal and vaginal capsules, nasally by means of a spray or into the eyes by means of liquid/gelatinous drops. The neurotrophic composition may also be formulated as creams or ointments for topical administration. The neurotrophic composition could be administered per os (e.g. capsules) depending on its composition, e.g. to do so all composition components must be absorbable by the gastrointestinal tract. For example, CP as such cannot be recommended for oral administration because, as a large molecule, it would not be intestinally absorbed. This may not however apply to smaller and/or functional derivatives of this protein provided their formulation is in absorbable forms (e.g. liposomes).

The neurotrophic composition of the invention may be administered alone or as part of a more complex pharmaceutical composition according to the desired use and route of administration. It may also be part of a commercial package containing as an active pharmaceutical ingredient ceruloplasmin or a functional derivative of ceruloplasmin, together with instructions for the use thereof. Anyhow, for preparing such compositions and packages, methods well known in the art may be used.

Ceruloplasmin or a functional derivative thereof could also be used in methods for culturing neuronal cells in vitro. By providing an effective amount of ceruloplasmin to in vitro cultured neuronal cells, it will induce the aggregation of neuronal cells and also promote the tissular organization of in vitro cultured neural tissues. Ceruloplasmin or a functional derivative thereof could thus be very useful for culturing neural tissues for transplant purposes.

As it will now be demonstrated by way of an example hereinafter, the composition of the invention possesses a strong neurotrophic activity, e.g. the capacity to promote the regeneration of neural tissues by modulating the association (aggregation) of neuronal cells cultured in vitro. Although any method and material similar or equivalent to those described herein can be used in the practice or testing of the present invention, the preferred methods and materials are described.

### EXAMPLE

### Modulation of Neuronal Organization by Ceruloplasmin

Abbreviations: BAEE, Nα-benzoyl-L-Arg-ethyl ester; CP, ceruloplasmin; ECM, extracellular matrix; GPI, glycosylphosphatidylinositol; α-MEM, alpha-modified Eagle's minimal essential medium; PBS, phosphate buffer saline; RA, retinoic acid; SBTI, soybean trypsin inhibitor; TIU, trypsin inhibitory unit; tPA, tissue plasminogen activator.

### 1. Introduction

1.1 Ceruloplasmin—a Multifunctional Copper Protein

A number of substances are known to influence neuronal development, including growth factors, components of the extracellular matrix (ECM), and cell adhesion and guidance molecules. Ceruloplasmin (CP) is a multifunctional copper-glycoprotein of 132 kDa produced by hepatocytes and found in plasma (1.5-3 μM). The protein is also synthesized in other tissues including the brain where a glycosylphosphatidylinositol (GPI)-anchored form is expressed by astrocytes (Patel and David, 1997; Patel and David, 2000). It is the major copper carrier in circulation, a scavenger of oxygen free radicals (OFR), and an enzyme displaying significant ferroxidasic and oxidasic activities. These functions are likely implicated in various physiological roles of the protein, such as regulation of copper and iron homeostasis (Bowman, 1993; Mukhopadhyay et al., 1998), induction of angiogenesis (McAuslan et al, 1983), possible. modulation of the acute phase response to inflammation (Bowman, 1993) and, demonstrated by our group, protection of heart (Chahine et al, 1991; Atanasiu et al, 1995; Dumoulin et al., 1996) and neurons (Paquin et al., 1999) against damages caused by oxidative stress conditions, and modulation of potassium ion channels (Wang et al., 1995).

1.1. 1 Ceruloplasmin and Human Pathology

Specific CP receptors have been found in mature erythrocytes (Barnes et al., 1984) as well as in membrane preparations obtained from aortic, cardiac, hepatic and cerebral tissues (Stevens et al., 1984; Orena et al., 1986; Frieden, 1986; Fischer and Goodie, 1994). However, the mode of interaction of CP with these receptors and the functional significance of this interaction, besides involving the transfer of copper to cells, remains unclear, suggesting the existence of still unknown properties of CP.

Inappropriate copper levels in nerve tissues, like those found in the diseases of Menkes (low copper levels) and Wilson (accumulation of copper), lead to severe neurological disorders and neurodegeneration (Wagooner et al, 1999). Aceruloplasminemia, a genetic deficiency of CP, causes severe intracellular iron accumulation in several organs, including the brain. In the latter organ, iron accumulation is associated with neurodegeneration, probably as a consequence of oxidative stress (Harris et al., 1995; Klomp and Gitlin, 1996; David and Patel, 2000). Altered brain iron metabolism and free radical injury are also associated with other neurodegenerative pathologies such as Parkinson's disease, Alzheimer's disease, amyotrophic lateral sclerosis and Hallervorden-Spatz' disease. Thus, it is possible that a lack of functional CP could contribute to syndromes of these diseases (David and Patel, 2000).

1.1.2 Ceruloplasmin Biochemistry

The “blue copper” center of CP has a characteristic absorption band at 610 nm. The protein contains six copper atoms per molecule. Three copper atoms are aggregated in a cluster which is the Blue-Copper center of CP. Two others form a diamagnetic pair, the last one is paramagnetic (detectable by electronic paramagnetic resonance or EPR).

An absorbency ratio A_{610 nm}/A_{280 nm}=0.040 or higher was considered in the literature as characteristic of a homogeneous standard pure enzyme. It was reported for CP a high susceptibility to proteolysis, and physiological properties influenced by the molecular integrity. Despite intensive research in various laboratories, many aspects of CP are still unclear. The protein has been the object of many controversies (originated from its high susceptibility to proteolysis) concerning the molecular characteristics and the copper content. Also controversial was its complex physiological role as antioxidant/prooxidant (Gutteridge, 1994; Chahine et al., 1991; Fox et al., 1995). Within the last decade, a continuously growing interest concerns the molecular mechanisms of protection and functions at cellular and tissular level, induced by CP.

It was recently shown that CP structure comprises six domains. Surprisingly, its configuration appears close to that of clotting Factors V and VII. However, the enigma is not ended. The intriguing fact is that CP receptors were identified, localized in tissues strongly involved in oxidative processes (heart) or sensitive to oxidative stress (brain: known to be damaged by the oxidative stress, especially in ageing). The presence of specific ceruloplasmin receptors with specific localization in aorta and heart, in brain, on erythrocytes and recently reported, on placenta (Fisher and Goodie, 1994; Barnes and Frieden, 1984; Orena et al., 1986, Stevens et al., 1984) is now established. Liver endothelium was shown to bind, transport and desialate CP, which is then recognized by galactosyl receptors of hepatocytes. Also, the production of CP by lung, nervous system (astrocytes, oligodendrocytes, Schwan cells, fibroblasts, leptomeningeal cells) etc. was shown. What is the real role of this CP synthesized in extra-hepatic tissues, is still to elucidate.

A questionable aspect is if CP (132 kDa) can be internalized as the whole molecule or as fragments. Chudej et al (1990) reported the transcytosis of exogeneous superoxide dismutase (SOD) and even of catalase (240 kDa) from coronary capillaries into dog myocytes. This is a particular case and a complete answer is not yet available for other cell or tissue types. In any case, an interaction of CP with cells was supposed.

1.2 A Single-step Chromatographic Method for the Fast Purification of Ceruloplasmin

Recently, a novel single-step chromatographic method has been reported for the fast purification of ceruloplasmin, a method leading to a purified, electrophoretically homogeneous protein (Mateescu et al., 1999). Ceruloplasmin is susceptible to proteolytic denaturation and this fast method therefore protects CP against such denaturation by decreasing time of eventual contact with proteolytic enzymes found in plasma or blood. The purification procedure is based on the highly selective retention of CP on Amino-ethyl (AE)-agarose. Using this procedure, it is possible to obtain CP preparations with ratio A₆₁₀/A₂₈₀=0.045-0.070 and a very high oxidase activity. The purification method permits to minimize the risk of protein degradation. In fact it is supposed, following a reexamination of CP spectral properties (EPR [Calabrese et al., 1988]), that CP purified using this procedure is closer to its real native structure than commercial CP obtained by other methods. This method allows to realize an original CP immobilization (Mateescu et al, 1988). The conjugation of CP with biocompatible polymers is important because the immobilized enzyme conjugates show sought-for advantages such as higher stability, lower antigenicity and possibility to continuous use in various devices of potential interest for bioimplants or for organ preservation in view of transplantation.

1.3 Presentation of the Study

Ceruloplasmin (CP) is an essential regulator of the metabolism of copper and iron ions which are necessary to sustain neuronal activity. CP modulates the activity of neuronal K⁺ channels, lack of functional CP causes neurodegeneration, and expression of CP and receptors for this protein has been reported in nervous system. All these findings have lead the present inventors to hypothesize that CP has an influence on neuronal development. The present inventors tested this hypothesis using the P19 mouse embryonal carcinoma cell line. These developmentally pluripotent cells differentiate to functional CNS neurons in vivo and in vitro when treated with retinoic acid (RA), and are thus considered as an excellent model of neuronal differentiation (McBurney, 1993; Finley et al, 1996; Jeannotte et al., 1997; Parnas and Linial, 1997; Cadet and Paquin, 2000). This work presents evidences that CP modulates the development of newly differentiated neurons and influences the pattern of organization of neuronal cells.

### 2. Materials and Methods

2.1 Purification, Heat-denaturation and Deglycosylation of Ceruloplasmin

Ceruloplasmin (CP) was purified from bovine plasma by fractionated precipitation with ammonium sulfate followed by chromatography on aminoethyl-agarose (Mateescu et al., 1999). The chromatographic material was obtained by treatment of cross-linked agarose beads (CL-SEPHAROSE™ 6B, Pharmacia, Upsala, Sweden) with chlorohydrate of 1-chloro-2-ethylamine (Sigma-Aldrich, Oakville, Ontario) as described (Mateescu et al., 1999). Purified CP was electrophoretically homogenous. Heat-denatured CP was obtained by heating a solution containing 2 mg/mL of the protein in 20 mM potassium phosphate buffer, pH 7.4, at 65° C., overnight; the solution was then concentrated ten fold by lyophilisation before being used with cells. Deglycosylated CP was obtained by incubating the native protein with N-glycosidase F (New England LabSystem, Beverly, Mass.) at a ratio of 3000 glycosidase units/mg CP, for 24 h, at 37° C., followed by chromatography on aminoethyl-agarose (Aouffen et al., 2001). The efficiency of deglycosylation was controlled by electrophoresis with the use of GELCODE GLYCOPROTEIN STAIN™ (Pierce, Rockford, Ill.), a specific dye for carbohydrates.

2.2 Cell Culture

P19 cells were cultured and differentiated as described by Jeannotte et al. (1997), with slight modifications. Undifferentiated P19 cells were propagated in complete medium containing alpha-modified Eagle's minimal essential medium (α-MEM; Gibco-BRL, Burlington, Ontario) supplemented with 10% heat-inactivated fetal bovine serum (Cansera International, Rexdale, Ontario), 2.5 U/mL penicillin and 2.5 μg/mL streptomycin (Sigma-Aldrich). Cells were maintained at 37° C. in a humidified atmosphere of 5% CO₂ and passaged every two days on non-coated tissue culture dishes. To achieve neuronal differentiation, P19 cells were seeded in bacteriological-grade petri dishes at a density of 0.9×10⁵ cells/mL, and grown as aggregates, during 4 days, in complete medium containing 0.5 μM RA (Sigma-Aldrich). At day 4, aggregates were dissociated by incubation for 3 min, at room temperature, with 0.025% trypsin (250 Nα-benzoyl-L-Arg-ethyl ester (BAEE) unit per 15×10⁶ cells; Sigma-Aldrich) in phosphate buffer saline (PBS) containing 1 mM EDTA, and digestion stopped by adding an equal volume of complete α-MEM medium. The resulting cellular suspensions were passaged by pipetting and subjected to two wash-and-centrifuge cycles, and individualized cells (neurons) were transferred (1500-1800 cells/mm²) to gelatinized tissue culture dishes or multiwell plates containing NEUROBASAL™ medium (Gibco-BRL) supplemented with B27™ supplement (Gibco-BRL) and 0.5 mM L-glutamine, but no RA. Gelatinization of culture surfaces was done by incubation with 0.1% gelatin for 0.5 h at 22° C., followed by several washes with PBS to remove unbound protein. For comparison purposes, solutions of 2% gelatin, 10 μg/mL fibronectin, 10 μg/mL laminin and 5 mg/mL CP (in this case, in a proportion of 50 μL per cm²) were also used for coating. Differentiation to fibroblasts was done by culturing P19 cells as monolayers, for 4 days, in complete medium containing RA. Cells were trypsinized every 2 days and replated in non-coated tissue culture dishes each time. From day 4 and after, cells were cultured in complete medium without RA. Monolayers of rat aortic smooth muscle cells (Wu and de Champlain, 1996) and rat hepatocytes (Haïdara et al., 1999) seeded on plastic were generously provided by Dr J. de Champlain (Université de Montréal) and by Dr F. Denizeau (Université du Québec à Montréal), respectively.

2.3 Treatment of Cells with CP and Other Agents

At 3, 24 or 48 h after plating, P19 neurons were incubated for 24 h in a fresh provision of supplemented Neurobasal medium not containing (control) or containing CP or another agent. Protease inhibitors were added 1 h before CP. Freshly individualized cells of the other cell types tested were let to adhere to culture surfaces, in their normal culture medium (containing serum), for times indicated: undifferentiated P19 cells and their fibroblast derivatives (both for 3 and 24 h), rat smooth muscle cells (7 days), and rat hepatocytes (3 h). Afterwards, cells were extensively rinsed with PBS and incubated for 24 h in supplemented Neurobasal medium in the absence or presence of CP. After treatment with CP or other agents, cells were examined for morphology, viability, ALAMARBLUE™ dye penetration and/or apoptosis, as indicated below. His₂Cu solution was prepared as per Sarkar et al. (1993) by dissolving 0.106 g anhydrous CuCl₂ and 0.244 g L-histidine in approximately 90 mL of 0.9% w/v NaCl. After adjustment to pH 7.4, the volume was completed to 100 mL with saline. The solution was then sterilized by filtration through a 0.22 μm-filter and stored at 4° C., protected from light. Laccase and soybean trypsin inhibitor (SBTI) were from Sigma-Aldrich, and albumin and aprotinin from Roche Diagnostics (Laval, Quebec). SBTI and aprotinin respectively had a specific activity of 70 and 200 trypsin inhibitory units (TIU)/mg as assayed with BAEE by the manufacturer. Bovine serum amine oxidase was purified as described by Mateescu et al., (1999).

2.4 Cell Morphology

Morphological analysis was done by inversion microscopy (NIKKON™ TMS), using phase-contrast objectives and a camera (NIKKON™ F70). Photographs were taken at 100× magnification with KODAK™ technical pan films and developed as 10.5×15 cm pictures. Morphological quantification of aggregation was done by placing a transparent grid made of 25 mm² squares over cell pictures and counting the squares which were more than 50% unoccupied by cells (aggregation increased the number of empty squares). Three different pictures were systematically obtained for each dish and an averaged number of empty squares from these pictures was used to characterize the dish. Surfaces not occupied by cells in treated cultures were expressed relatively to those of control (untreated) cultures. Because of its simplicity and rapidity, this grid method was adopted after correlation of the results by image analysis with UN-SCAN-IT™ software (SilkScientific Inc.).

2.5 Cell Viability

Cells were rinsed with warm PBS and incubated in PBS containing 0.25% trypsin (1000 BAEE units/mg) and 1 mM EDTA (1 mL/well of 6-well plates) for 10 min at 37° C. Digestion was stopped by addition of an equal volume of complete α-MEM medium. Cells were passaged by pipetting, stained by addition of another volume of a solution containing propidium iodide (20 μg/mL) and acridine orange (3 μg/mL) in PBS, and counted with a hemacytometer under a fluorescent microscope set up to excite for fluorescein. Dead and live cells exhibited a red and green fluorescence respectively.

2.6 ALAMARBLUE™ Dye Penetration

Day 4 P19 neurons were plated in 24- or 48-well plates in prevision of dye penetration analysis. After treatment with CP or other agents, cells were incubated for 5 h with 500 μL of a solution containing one part of ALAMARBLUE™ (Medicorp Inc., Montreal, Quebec, Canada) and nine parts of fresh supplemented Neurobasal medium. This dye is reduced to a fluorescent derivative by metabolic activity of living cells. At the end of incubation, the fluorescence of the culture medium was monitored at 590 nm, using 540 nm as the excitation wavelength.

2.7 Apoptosis Analysis

Day 4 P19 neurons were plated in 24-well plates in prevision of apoptosis evaluation. Quantitation of mono- and oligonucleosomes was done on cell extracts with a NUCLEOSOME ELISA KIT™ (Oncogene Research Products, Calbiochem, Boston). Cells were suspended by pipetting in 350 μL of lysis buffer (provided with kit and freshly complemented with 0.2 mM phenylmethylsulfonylfluoride), incubated for 30 min on ice and centrifuged. Cell extracts (supernatants) were frozen at −20° C. for at least 18 h and then assayed for nucleosome as per manufacturer's instructions. Apoptosis was also evaluated under the microscope with Hoechst 33258 dye (Sigma-Aldrich). Neuronal monolayers or their aggregates were directly incubated for 15 min at room temperature in presence of the dye added at a final concentration of 50 μg/mL (from a stock solution of 1 mg/mL) in culture medium. Neurons were then washed twice with PBS, immersed in this buffer and observed by fluorescence microscopy to detect cells containing condensed chromatin. Alternately, cells were trypsinized with 0.25% trypsin-1 mM EDTA before treatment with Hoechst 33258 dye to facilitate the counting of apoptotic cells in overall populations.

2.8 Electrophysiological Studies

Voltage-dependent K⁺ channel currents of P19 neurons were recorded using the whole-cell configuration of the patch-clamp techniques, as described for neuroblastoma cells (Wang et al., 1995).

2.9 Detection of Proteolytic Activity

Evaluation of proteolytic activity in CP preparations or in cell culture media was done with resorufin-labeled casein (universal protease chromogenic substrate; Roche Diagnostics). Aliquots (100 μL) of CP solutions (about 13 mg/mL) or of culture media of cells after 24 h treatment with and without CP were incubated at 37° C. for various times (up to 22 h), with 50 μL of resorufin-labeled casein solution provided by the manufacturer and 50 μL of 0.2 M Tris-HCl, pH 7.8 containing 0.02 M CaCl₂. The reaction was stopped by adding 480 μL of 5% trichloroacetic acid. After 10 min of incubation at 37° C. and 5 min of centrifugation, 400 μL of the resulting supernatants were mixed with 600 μL of 0.5 M Tris-HCl, pH 8.8, and absorbance read at 574 nm. Alternately, resorufin-labeled casein was added to culture media (50 μL substrate for each 150 μL supplemented NEUROBASAL™ medium) while cells were being treated with CP. Culture media were removed at various times (up to 24 h) and subjected to acid precipitation and spectrophotometric measurement as indicated above. Trypsin (10100 U BAEE/mg; Sigma-Aldrich) was used as a standard.

### 3. Results

3.1 CP has a Saturable Pro-aggregative Effect on Neurons

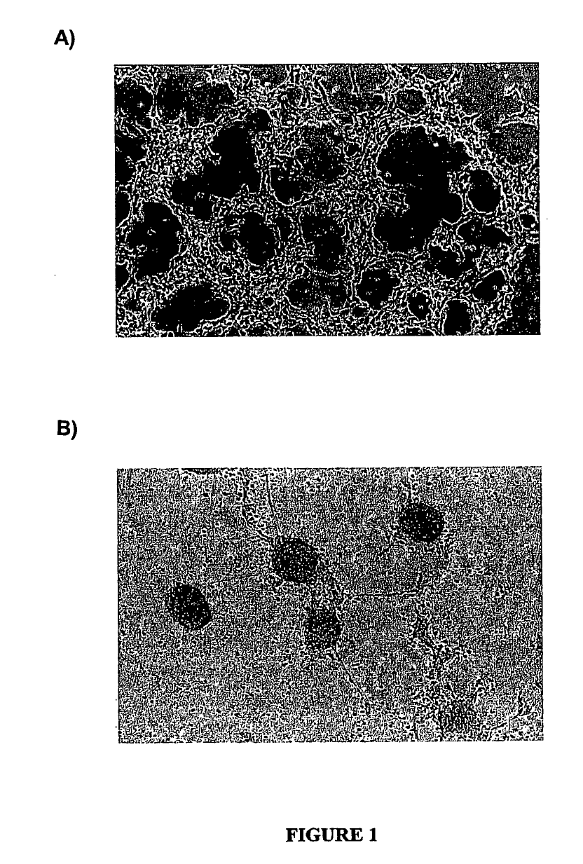

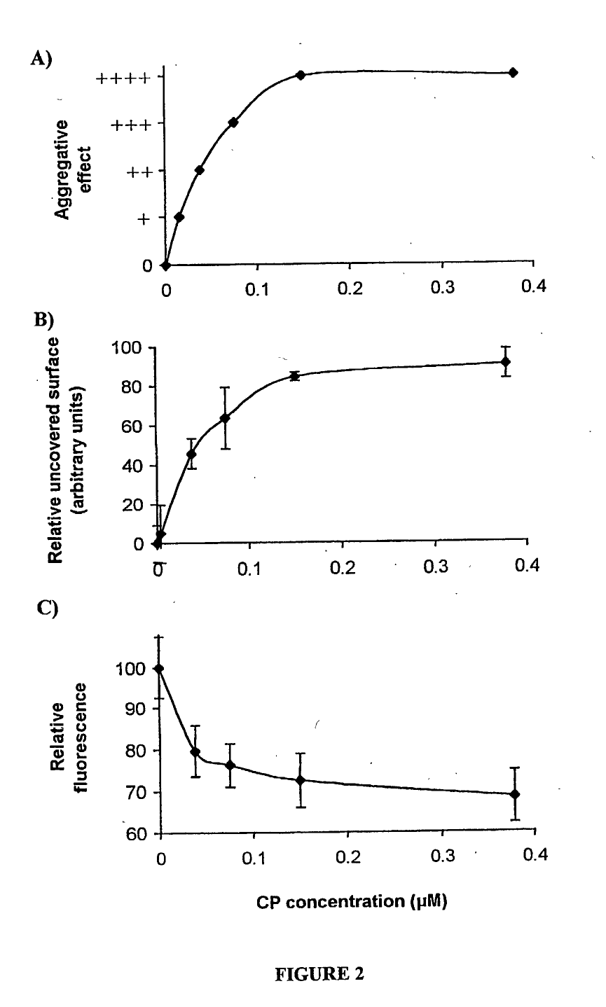

P19 cells at day 4 of neuronal differentiation, at end of exposure to retinoic acid, have already begun to express neuronal markers (McBurney, 1993; Jeannotte et al., 1997). They then continue to mature in a stepwise manner. For example, day 8 neurons synthesize and store bioactive neuropeptides but become able to release these substances in response to membrane depolarization by K⁺ only around day 12 (Cadet and Paquin, 2000). It is possible that delay in apparition of evocable secretion in neurons may not be as evident in vivo as in vitro due to differences in environmental conditions, such as the presence of specific trophic factors. One candidate trophic factor could be CP since it can modulate the activity of K⁺ channels (Wang et al., 1995), could influence the functionnality of copper-dependent enzymes belonging to neurosecretory pathways (Hartmann and Evenson, 1992; Waggoner et al., 1999) and, moreover, could be in contact with neurons via its expression at the surface of glial cells (Patel and David, 1997; Salzer et al., 1998). We therefore exposed day 4 P19 neurons to 3.8 μM CP, a top concentration value for the circulating protein under normal physiological conditions. Unexpectedly, we observed a strong aggregative effect of the protein on cells. Indeed, while neurons cultured in the absence of CP formed well spread cell monolayers, those treated for 24 h with the protein formed spherical and compact cell aggregates that adhere loosely to the culture surface but still exhibited interconnecting neurites (FIG. 1). Visual examination showed that the pro-aggregative effect of CP on culture morphology was concentration-dependent up to 0.15 μM of the protein and leveled at higher concentrations (FIG. 2A). Aggregation was stable even at concentrations as high as 3.8 μM (not shown). Aggregation was quantified by two other methods. First, since packing increased empty space between cells, we calculated the augmentation in culture surfaces not occupied by cells using a transparent grid placed over photomicrographs. The concentration-response curve obtained with this technique (FIG. 2B) matched the results of visual appreciation (FIG. 2A). The concentration of CP necessary to achieve 50% of the aggregative effect was approximately 0.05 μM with both methods. The viability dye ALAMARBLUE™ can penetrate cells where it is reduced by cellular metabolism. Since CP treatment did not affect cell viability (next section) but caused a decrease in dye reduction by cells (FIG. 2C), it can be concluded that aggregation induced by CP prevented access of the dye to cells. Fluorescence monitoring of dye reduction confirmed the concentration-dependent and saturable pro-aggregative effect of CP (FIG. 2C). CP was found not to interfere in reduction/oxidation of ALAMARBLUE™ dye.

3.2 CP does not Compromise Neuronal Viability

CP was found to be toxic to isolated rat hearts when present at concentration equal to or greater than 4 μM in perfusing buffer (Chahine et al., 1991; Atanasiu et al., 1995) and lethal to NIE-115 neuroblastoma cells if used at more than 1 μM during patch-clamp manipulations (Wang et al., 1995). To determine if the pro-aggregative action of CP was the result of some cytotoxic properties of the protein and/or if aggregation could eventually promote cell death even though CP was not toxic by itself, we evaluated cell viability of P19 neurons exposed to CP. Once aggregated by treatment with CP, neurons were individualized by trypsinisation and their viability assessed with the use of the fluorescent dyes acridine orange and propidium iodide. Even after exposure to CP concentrations up to 7.6 μM, almost all cells incorporated acridine orange only and not propidium iodide, indicating intact plasma membranes and viability (not shown). Beside necrosis, apoptosis is another cause of cell mortality. This genetically programmed cell death naturally occurs during development of the nervous system with the role of removing neurons produced in excess (Honig et al., 2000). Nucleosome assay indicated that CP concentrations up to 3.8 μM did not induce internucleosomal DNA fragmentation characteristic of apoptotic events since treated and control cells showed a similar low nucleosome level (not shown). In contrast, 15-min irradiation of P19 neurons with ultraviolet light (followed by 24 h cultivation under normal conditions) and incubation of hepatocytes for 24 h in the presence of 0.16 ng/mL β-transforming growth factor (β-TGF), two treatments known to induce apoptotic DNA fragmentation, increased nucleosome level about 5-fold (not shown). Fluorescent microscopy of cells labeled with Hoechst 33258 dye showed that less than 5% of P19 neurons treated with CP were fluorescent (not shown). A similar response was seen with non treated control cells, indicating that CP treatment did not induce chromatin condensation, another sign of apoptosis (Honig et al., 2000). Both nucleosome and Hoechst dye assays demonstrated the absence of a pro-apoptotic effect of CP on P19 neurons.

3.3 Morphologic Effect of CP on Ageing Neurons and on Non-neuronal Cells

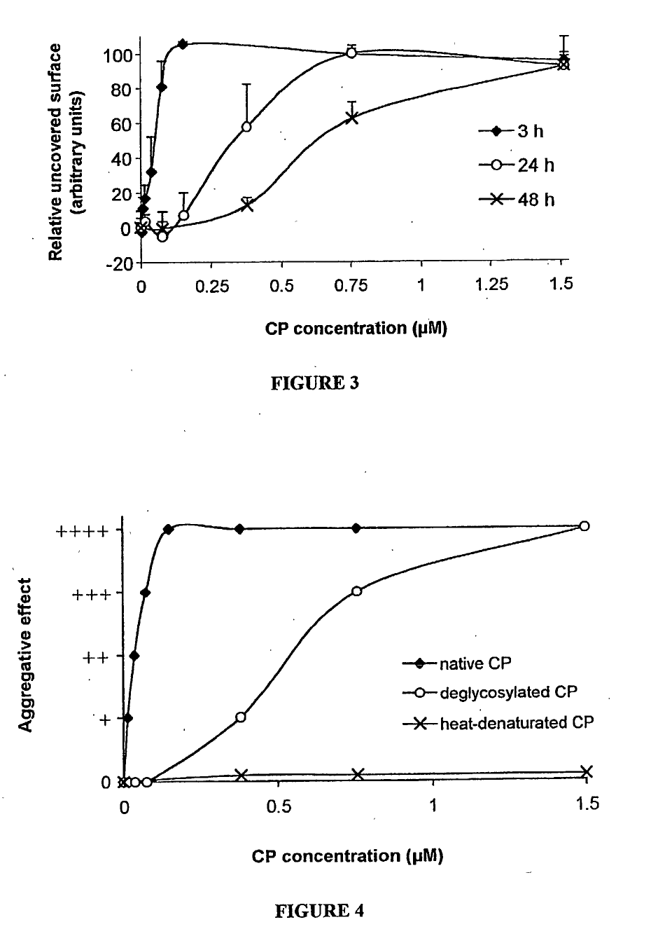

Since newly differentiated P19 neurons continue to mature in vitro, they could exhibit an age-dependent response to the pro-aggregative influence of CP. We thus compared the response of P19 neurons incubated with the protein at different times after plating. In the absence of CP, P19 neurons had begun to adhere to the substratum 3 h after plating but had not yet extended neurites, while 24 h later they had spread over the culture surfaces and already exhibited processes developing into an elaborated network. When CP was administered 24 h or 48 h after plating instead of 3 h, neurons still aggregated during a 24 h incubation with the protein, despite the presence of already grown neurites. However, ageing decreased neuron sensitivity towards CP action. Indeed, the concentration of CP to achieve half maximal aggregation increased from 0.05 to 0.33 and 0.62 μM, respectively (FIG. 3). Interestingly, aggregates were often interconnected to each other by long and thick processes (as in FIG. 1B), suggesting that CP does not cause neurite retraction by itself. Addition of CP more than 72 h after plating did not induce aggregation nor neurite retraction (not shown). We also exposed non-neuronal cell types to 3.8 μM CP, a saturating pro-aggregative concentration with neurons. Contrarily to what was observed with P19 neurons, CP did not display pro-aggregative effects on undifferentiated P19 cells, P19 cells differentiated to fibroblasts, rat aortic smooth muscle cells (in this case, no effect was seen even after 48 h treatment) and rat hepatocytes, indicating a cell type dependent phenomenon (not shown).

3.4 Culture Substratum does not Seem to Influence CP-induced Neuronal Aggregation

In vivo, cell motility and adhesion are controlled by interactions of cell surface proteins with components of extracellular matrix (ECM). Considering that the aggregative action of CP on newly differentiated neurons may be due to an effect on cell-matrix interactions, we examined if the type of matrix used to cultivate cells could influence this action. P19 neurons were plated on culture surfaces coated with gelatin, with fibronectin or laminin (two matrix proteins found abundantly in brain), or with poly-D-lysine, a synthetic polypeptide often used to promote cell adhesion in vitro. P19 neurons extensively aggregated (++++) on each culture substratum during incubation with 3.8 μM CP (not shown). Also, when CP itself was tested as a coating material, it had an aggregative effect similar to that of soluble CP and failed to promote monolayer formation (not shown). It appears that CP-induced neuroaggregation was independent of matrix type.

3.5 Denatured CP, Copper Ions, other Copper Enzymes and Serum Proteins do not Induce Neuronal Aggregation

Several but not all actions of CP strictly depend of its native form. For example, native but not heat-denatured CP has ferroxidase and oxidase activities, protects isolated rat hearts against oxidative injury induced by electrolysis or by ischemia-reperfusion (Chahine et al., 1991; Mateescu et al., 1995; Dumoulin et al., 1996), or modulate the activity of neuronal K⁺ channels (Wang et al., 1995). In contrast, heat-denatured CP is still effective as a class III antifibrillatory agent on heart (Atanasiu et al., 1996) or as a free radical scavenger in vitro (Atanasiu et al., 1998). CP loses its blue color upon heat denaturation (Dumoulin et al., 1996) and as much as 50% of its copper content. Deglycosylation does not compromise the enzymatic activities of CP nor its ex vivo cardioprotective and neuronoprotective actions in oxidative stress (Aouffen et al., 2001), but removal of sialic acid residues was reported to severely diminish half-life of the protein in circulation (Morell et al., 1968). We thus investigated the importance of the structural integrity of the protein in inducing neuronal aggregation. As showed in FIG. 4, heat-denatured CP failed to induce aggregation even at high doses of the protein. In contrast, deglycosylated CP was still effective although less potent, with an approximately 10-fold increase in concentration required to induce half-maximal aggregation (FIG. 4). The results indicate that the protein must maintain overall or some local polypeptidic conformation to exert its aggregative action, and the presence of carbohydrates, although not essential, could confer stability to the protein and/or could have a potentiating effect, perhaps increasing binding to potential receptors. Gamma irradiation of CP with ⁶⁰Co decreased potency of the aggregative action of CP in a dose-dependent manner: 2.3 μM CP irradiated with 3 and 6 kGy respectively induced a (++++) and a (+++) aggregative effect on neurons while irradiation with 10 kGy abolished aggregation. Irradiation could induce cross-links between CP molecules and these links could inhibit the activity of CP, change its conformation and/or hide specific interaction sites.

Bovine CP, like its human counterpart, contains six copper ions per polypeptide chain (Calabrese et al., 1981, Zaitseva et al., 1996). Additional copper atoms could also be loosely bound to both proteins. A possible mechanism of the aggregative effect of CP on neurons could be related to copper ions transferred to cells by means of CP-neuron interactions or released from the protein into the culture medium. Therefore, the effects of copper salts on neurons was studied and the results are presented hereinbelow in Table 1.

TABLE 1
Effect of copper ions, copper enzymes and serum proteins on the
morphology of P19 neurons.

| Agent | Concentration (μM) | Morphological change |
|---|---|---|
| CuSO₄ or CuCI₂ | 1.14 to 49.2 | no aggregation |
| | 246 | cell death |
| (His)₂Cu complex | 23 to 390 | no aggregation |
| Laccase | 0.79 to 7.9 | no aggregation |
| Bovine serum amineoxidase | 0.28 to 0.56 | no aggregation |
| | 1.1 | cell death |
| Bovine serum albumin | 0.3 to 7.5 | no aggregation |
| Day 4 P19 neurons were plated on gelatin-coated dishes and incubated for 24 h with different concentrations of the indicated agent. Morphology of cell | | |
| # cultures was examined under the microscope. Cell death was characterized by cellular shrinkage and lysis. Since there are six coppers in the structure of | | |
| # bovine CP, a concentration of 22.8 μM copper ions simulated a complete release of all copper atoms from 3.8 μM CP. | | |

As shown in Table 1, treatment of cells with copper salts (CuSO₄ and CuCl₂) at copper concentrations equivalent to those found in CP did not induce neuronal aggregation Histidine is a physiological copper ligand and transporter found in normal human serum (Sarkar et al., 1993). Like the three type-I copper bound to CP, copper complexed to histidine as His₂Cu is blue. The complex was shown to allow effective Cu²⁺ uptake by brain tissues and incorporation of this ion into intracellular cuproproteins such as superoxide dismutase (Dameron and Harris, 1987; Katz and Barnea, 1990). The complex was also reported to interact with potential CP receptor/copper transporter in human placental vesicles (Hilton et al., 1995). His₂Cu at concentrations equivalent to or even higher than that of copper in CP did not induce neuronal aggregation (Table 1). Copper salts were toxic to P19 neurons at high micromolar concentration while His₂Cu was not (Table 1), underlining the biocompatibility of the “protein-like” copper complexed to histidine. Results thus show that neither free nor histidine-coordinated copper ions were pro-aggregative by themselves. The action of CP on P19 neurons was also compared to that of other copper enzymes and serum proteins. Laccase (64 kDa), structurally similar to CP by having a blue copper center (Messerschmidt and Huber, 1990), was not pro-aggregative (Table 1). Serum amine oxidase (180 kDa), a circulating non-blue copper protein shown to modulate the activity of K⁺ channels (Wu et al., 1996), did not induce aggregation either (Table 1). It even caused cell death when used at 1.1 μM (Table 1) while CP up to 7.6 μM was not detrimental to cells. Cell death can be related to the release of cytotoxic H₂O₂ and aldehydes, products of amine oxidase activity (Averill-Bates et al., 1993). Serum albumin which can transport copper ions among other substances and act as an antioxidant, although less potently than CP (Dumoulin et al., 1996; Atanasiu et al., 1998), also failed to induce-aggregation (Table 1). In addition, albumin at 3.6 μM did not inhibit CP-induced neuroaggregation (not shown). The aggregative action was thus specific to CP and not due to a general protein or copper effect.

3.6 CP-induced Neuronal Aggregation is Inhibited by Serine Protease Inhibitors

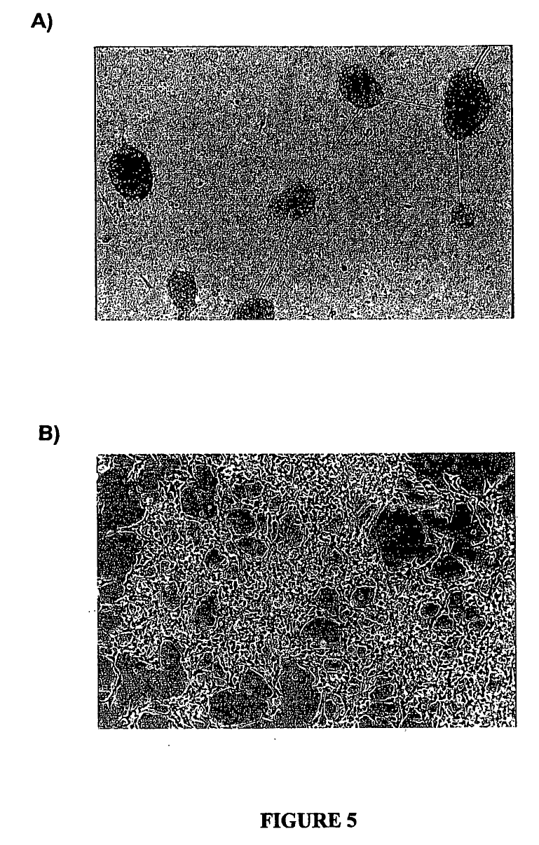

During nervous system development, extracellular serine proteases have been implicated in processes such as neuronal migration, axon genesis and formation of mature synaptic connections (Yoshida and Shiosaka, 1999). For example, thrombin is known to inhibit and reverse neurite outgrowth in cell culture, and plasmin, to induce cell body rounding and increase neuronal migration (Shea and Beermann, 1992). Thrombin or a thrombin-like protease as well as plasminogen, the source of plasmin, and its activating proteases urokinase and tissue plasminogen activator (tPA) were all shown to be expressed in developing brain or in various neuronal populations (Shea and Beermann, 1992; Yoshida and Shiosaka, 1999). Moreover, neuronal migration was recently demonstrated to be retarded in mice lacking the tPA gene (Seeds et al., 1999). On the other hand, CP is composed of a triplicated A domain that shows approximately 30-35% sequence identity with the same motif found in blood coagulation Factors V and VIII which either assist the cleavage of prothrombin to thrombin or serve as a thrombin substrate (Pemberton et al., 1997). We thus used aprotinin and SBTI, two inhibitors that do not diffuse across cell membranes, to determine if pericellular serine proteases like thrombin or plasmin/plasminogen activators could be involved in the pro-aggregative effect of CP. Both inhibitors (at 6-7 trypsin inhibitory U/mL each) completely hindered CP-induced neuronal aggregation, as assessed by morphology (FIG. 5) and dye penetration analysis (not shown). It can be concluded that a proteolytic event occurred with induction of aggregation by CP and likely involved a pericellular serine protease. Complete inhibition by SBTI and aprotinin and the fact that thrombin is not inhibited by these inhibitors (Barrett and McDonald, 1980) tend to eliminate thrombin to the profit of plasminogen activator(s)/plasmin, or another serine protease, as an active agent in CP-induced neuroaggregation. Expression of tPA has been reported in the P19 cell model (Mummery et al., 1987). Neither serum nor CP preparations were a source of proteases for neurons, since serum was omitted in neuron cultures and CP preparations had no detectable proteolytic activity as assayed with the general substrate resorufin-labeled casein and no gelatinase activity by gelatin zymography (not shown). The casein assay detected only low levels of proteolytic activity in culture media conditioned by cells and assayed in absence or presence of cells (10⁵ cells exhibited a pericellular proteolytic activitiy approximately equivalent to that of 0.004 BAEE unit of trypsin over 24 h), and levels were not modified by addition of CP (not shown). In all cases, aprotinin and SBTI completely abolished hydrolysis of resorufin-labeled casein (not shown). Thus, CP-induced neuroaggregation seemed involving a pericellular (secreted or associated to cell surface) protease but apparently not an upregulation of proteolytic activity, at least as measured with casein.

3.7 Electrophysiology Studies

In view that CP was shown to induce membrane depolarization in cultured neuroblastoma cells by acting on K⁺ channels (Wang et al., 1995) and that long term depolarizing conditions such as those created by high concentrations of K⁺ were found to be important for the maturation of some neurons in culture (Damgaard et al., 1996: Muller and Yool, 1998), we tested whether a potential effect of CP on the polarization status of membranes of P19 neurons could be involved in mediating cell aggregation. The fact that membrane depolarization combined to calcium influx was shown to cause the release of tPA by pheochromocytoma PC12 cells (Gualandris et al., 1996) was another,pertinent consideration since it raised the possibility of a link between CP aggregative effect, neuronal behavior and serine proteases. When P19 neurons at day 5, 6, 7 and 8 of differentiation were analysed by patch-clamping using the whole-cell configuration, they were found to be electrophysiologically active since membrane depolarization increased the voltage-dependent transmembrane K⁺ currents (not shown). Addition of CP to the perfusing buffer had no effect on the voltage-dependent K⁺ channel currents in these cells, indicating that early steps responsible for the aggregative action of CP did not apparently involve modulation of K⁺ channels. Different responses of K⁺ channels in P19 neurons and neuroblastoma cells (Wang et al., 1995) with respect to membrane depolarization by CP could be due to differential expression of K⁺ channels and/or the extent of cell maturation.

### 4. Discussion

This disclosure demonstrates, for the first time, that CP has a pro-aggregative action on newly differentiated neurons in vitro, adding to the multifunctional character of the protein. This action is specific in several aspects. The effect was concentration-dependent and saturable, indicating that it must obey to a definite mechanism (FIG. 2). Other copper enzymes and plasma proteins, like laccase, serum amine oxidase and albumin which share some properties with CP, all failed to induce neuronal aggregation (Table 1), thus eliminating the possibility of a general protein effect. Moreover, denaturation of CP by heat completely prevented aggregation, deglycosylation decreased potency of the aggregative effect and free copper ions at concentration found in the protein were not aggregative by themselves (FIG. 4 and Table 1), indicating the necessity of a native or a particular conformation of CP for aggregation. Intact copper center(s) and enzyme activity could also be required for aggregation because heat causes a partial release of copper ions and abolishes ferroxidase and oxidase activities. Finally, the aggregative effect appears to be cell-type specific since it was only observed in differentiated P19 neuronal populations, but not in cultures of undifferentiated P19 cells or those of fibroblasts, hepatocytes or aortic smooth muscle cells. However, CP was reported to reduce adhesion of leucocytes and BHK cells on plastic as well as leucocyte adhesion on endothelial cells (Curtis and Forrester, 1984; Broadley and Hoover, 1989), but reduced adhesion was seen at higher concentrations than those promoting neuronal aggregation. The specific and potent aggregative action of CP on neurons in conjunction with the recent demonstration that the protein is synthesized in nervous system (David and Patel, 2000) raise the possibility that CP may participate in tissue organization of that system, more especially perhaps but maybe not exclusively during development.

CP likely has a supportive role for mature neurons by regulating the transport/metabolism of copper and iron ions required by several neuronal enzymatic activities. Other roles of CP with neurons have also been proposed, including modulation of K⁺ channels (Wang et al., 1995) and protection against oxidative stress (Chahine et al., 1991; Aouffen et al., 2001). This report shows that, in addition, CP has a pro-aggregative influence on newly differentiated neurons (FIGS. 1 and 2). Aggregation being not the result of necrosis or apoptosis, most conceivably involved a direct or indirect action of CP on cell-cell and/or cell-matrix contacts. Interestingly, potency of the aggregative effect decreased while neurons were ageing in culture and had extended neurites (FIG. 4). The aggregative action of CP and its apparent temporally regulated character can have relevance to the stepwise establishment of the laminar architecture and neuronal connectivity of the developing brain. Indeed, the distinct functional areas of brain originate from an array of cellular layers assembled during development (Hatten, 1999). After differentiation, immature neurons migrate radially from germinative zones to neural laminae, and tagentially across laminae to form compact neuronal layers. Once at destination, they grow axons to establish contact and form synapses with targets. Neurons migrate along glial fibers (the counterparts of astrocytes in the embryonic brain) and on neuritic cables assembled by precedent migratory neurons (Hatten, 1999). It is thus tempting to propose that neuronal migration along glial fibers could implicate interaction between potential CP receptors at the surface of neurons and CP molecules anchored on fibers by a GPI group, similar to CP-GPI molecules expressed by mature glial cells (Patel and David, 1997; Salzer et al., 1998). The fact that CP can induce aggregation of neurite-bearing neurons and at the same time leave long neuritic connections between aggregates is also compatible with the idea of CP assisting neuron migration on neuritic cables. CP can influence neuron-neuron interactions (this work) and has the potential to mediate interactions of neurons with astrocytes, Schwann cells and brain fibroblasts since these non-neuronal cells were shown to express CP at their surface (Patel and David, 1997; Salzer et al., 1998; David and Patel, 2000). It is not known yet whether there are CP receptors on P19 neurons but membranes obtained from brain contained specific binding sites for the protein (Orena et al., 1986). Moreover, immunocytochemical studies done to test integrity of the fetal blood-brain barrier have revealed association of CP with neurons in the developing human brain (Mollgard et al., 1988). CP could also be involved in the establishment of neuron-cardiac muscle interactions because it modulates the electrophysiological properties of isolated heart (Atanasiu et al., 1996) and CP receptors were reported to exist in aorta and heart membrane preparations (Stevens et al., 1984). CP could have an initiating and transitory role in the establishment of cell contacts since in a coculture study with Schwann cells and neurons, the protein was reported to initially concentrate at sites of Schwann cell contact with neurons and was then downregulated (Saizer et al., 1998). Concentrations of CP inducing neuroaggregation in vitro (50% aggregation at 0.05 μM) were much lower than those found in plasma (1.5-3 μM) but similar to those found in CSF (about 0.01 μM; Hartard et al., 1993). These comparisons and the fact that expression of CP at the surface of glial cells can provide locally high concentrations of the protein in the vicinity of neurons would indicate that conditions exist in vivo to support the neuroaggregative activity of CP.

In addition to a potential role in neuronal development, the aggregative effect of CP could have significance in neurodegenerative pathologies. Regional increases in CP expression in brain of people suffering from various degenerative diseases, have been interpreted as possibly reflecting a defense mechanism against oxidative stress or inflammation (Castellani et al., 1999; David and Patel, 2000). In view of its aggregative properties, CP could also participate in the reconstruction of tissues desaggregated by disease. By therapeutically sustaining tissue reorganization, CP may additionally help stabilizing synaptic connections. In neuronal regenerating strategies, CP would thus beneficially assist the action of neurotropins—such as nerve growth factor (NGF) and brain-derived neurotrophic factor (BNDF)—that were shown to stimulate neurite sprouting and synaptic rebuilding (Lindvall et al., 1994). Even though this protein is already produced in brain, there is still an important reason to provide exogenous CP to persons suffering from neurodegenerative diseases. Indeed, oxidative stress, often associated with these diseases, could damage CP which we showed to be a target of reactive oxygen species in vitro (Aouffen et al., 2001; Paquin et al., 1 999).

The mechanisms underlying the aggregative action of CP on newly differentiated P19 neurons remain to be elucidated. As mentioned before, receptors could be involved but their existence on neurons is still to be demonstrated. CP conformation had a determinant role and glycosylation was also influential, two observations compatible with a receptor-mediated mechanism. In addition, neuroaggregation occurred at CP concentrations within the range of Kd values (0.01-10 μM) obtained from binding studies done on various tissue and cell preparations (Barnes and Frieden, 1984; Stevens et al., 1984; Kataoka et al., 1985; Puchkova et al., 1997). Potential neuronal receptors may differ from hepatic receptors since CP had no aggregative effect on hepatocytes. Copper ions have a recognized participation in angiogenesis and were shown to induce neovascularization in the rabbit cornea pocket assay and migration of bovine aorta endothelial cells at low micromolar concentrations (McAuslan et al., 1983). Although free copper ions were not pro-aggregative with neurons, they could still have a role in aggregation via CP-mediated transfer across neuronal membranes.

In accordance with the importance of pericellular serine proteases in regulation of developmental processes (Yoshida and Shiosaka, 1999), specific inhibitors of this class of proteases hindered CP-induced neuronal aggregation. Complete inhibition by aprotinin and SBTI would favor plasminogen activator(s)/plasmin over thrombin-like protein as potential candidate proteases. CP could upregulate/activate a protease acting on trophic factors, receptors and/or adhesion molecules. In line with these possibilities, Factors V and VIII structurally homologous to CP can act as co-factors in the proteolytic cascade of blood coagulation, tPA can proteolytically activate pro-hepatocyte growth factor/scatter factor (HGF/SF) in brain (Thewke and Seeds, 1999), and CP binding to erythrocytes seems to require a limited proteolysis of its receptors (Puchkova et al., 1991). No upregulation of proteolytic activity was observed in culture media assayed with the resorufin-labeled casein substrate. This could be due to detection limit of the assay but, alternately, it could suggest that CP may interfere in a signalling cascade involving the participation of a protease, or a CP fragment generated by constitutively expressed protease(s) may be responsible for the aggregative effect of the protein. There are examples of bioactive peptides released from large blood-derived proteins by proteolysis, such as fragment K5 from plasminogen or hemorphins from hemoglobin (Cao et al., 1999; Fruitier et al., 1999). In vivo, CP aggregative action may thus be modulated by protease inhibitors as well as chemotactic molecules. Finally, although this study was initiated with the assumption that CP can influence maturation of P19 neurons via an action on K⁺ channel activity, patch-clamp analysis indicated that initiation of neuroaggregation by CP seems not involving such channels.

### 5. Conclusive Remarks

CP was shown to have a pro-aggregative action on newly differentiated neurons. Although mechanisms responsible for aggregation are not known yet, this action appears to be a specific and reproducible property of the protein. The decreasing sensitivity of neurons towards CP aggregative effect with maturation suggests that the protein could have a role in early organization of the developing nervous system. However, CP could also assist efforts of the organism to rebuild neuronal organization after nerve damage since rebuilding may involve steps/molecules that normally act in development. The neuroaggregative action of CP adds to other properties of the protein as a modulator of iron/copper metabolism, an antioxidant, a protector of heart and neurons, and a modulator of neuronal and cardiac functions. Existence of potential receptors of CP in nervous system raises the possibility that CP can also be used as a drug carrier to neurons, thus opening the way for the design of a very novel therapeutic tool. The specific delivery of therapeutic drugs to target tissues is a mean to help prevent undesired spreading of drugs and secondary effects. In the case of CP (or a bioactive derivative), the drug carrier could enhance the drug effects by its own therapeutic action.

### 6. REFERENCES

Throughout this disclosure, reference is made to a number of articles of scientific literature which are listed below and incorporated herein by reference:

Aouffen M, Paquin J, De Grandpré E, Nadeau R, Mateescu MA (2001) Biochem Cell Biol, in press.

Atanasiu R, Chahine R, Mateescu M, Nadeau R (1995) Can J Physiol Pharmacol 73:1253-1261.

Atanasiu R, Gouin L, Mateescu M A, Cardinal R, Nadeau R (1996) Can J Physiol Pharmacol 74:652-656.

Atanasiu R L, Stea D, Mateescu M A, Vergely C, Dalloz F, Briot F, Maupoil V, Nadeau R, Rochefte L (1998) Mol Cell Biochem 189:127-135.

Averill-Bates D A, Agostinelli E, Przybytkowski E, Mateescu M A, Mondovi B (1993) Arch Biochem Biophys 300:75-79.

Barnes G, Frieden E (1984) Biochem Biophys Res Commu 125:157-162.

Barrett A J, McDonald, J K (1980) Mammalian proteases: A Glossary and Bibliography. London, England: Academic Press.

Bowman B H (1993) Hepatic plasma proteins: mechanisms of function and regulation. San Diago, Calif.: Academic Press Inc.

Broadley C, Hoover R L (1989) Am J Pathol 135:647-655.

Cadet N, Paquin J (2000) Dev Brain Res 120:211-221.

Calabrese L, Malatesta F, Barra D (1981) Biochem Int 199:667-673.

Calabrese L, Mateescu M A, Carbonaro M, Mondovi B (1988) Biochem Internatl 16:199-208.

Cao R, Wu H L, Veitonmäki N, Linden P, Farnebo, J, Shi G Y, Cao Y (1999) Proc. Natl. Acad. Sci. USA 96:5728-5733.

Castellani R J, Smith M A, Nunomura A, Harris P L R, Perry G (1999) Free Rad Biol Med 26:1508-1512.

Chahine R, Mateescu M A, Roger S, Yamaguchi N, De Champlain J, Nadeau R (1991) Can J Physiol Pharmacol 69:1459-1464.

Chudej L L, Koke J R, Bittar N (1990) Cytobios 63:41-53.

Curtis A S G, Forrester J V (1984) J Cell Sci 71:17-35.

Dameron C T, Harris E D (1987) Biochem J 248:663-675.

Damgaard I, Trenkne, E, Sturman J A, Schousboe A (1996) Neurochem Res 21:267-275.

David S, Patel B N (2000) Advances in structural biology (JAI Press Inc.) 6:211-237.

Dumoulin M J, Chahine R, Atanasiu R, Nadeau R, Mateescu M A (1996) Arzneimittel-Forsch/Drug Res 46:855-861.

Finley M F, Kulkarni N, Huettner J E (1996) J Neurosci 16:1056-1065.

Fischer A C, Goodie C A (1994) Prep Biochem 24:151-165.

Fox P L, Mukhopadhyay C, Ehrenwald E (1995) Life Sci 56:1749-1758.

Fruitier I, Garreau I, Lacroix A, Cupo A, Piot, J M (1999) FEBS Lett 447: 81-86.

Goldstein I M, Kaplan H B, Edelson H S, Weissmann G (1982) Ann N Y Acad Sci 92:360-369.

Gualandris A, Jones T E, Strickland S, Tsirka S E (1996) J Neurosci 16:2220-2225.

Gutteridge J M (1994) Ann NY Acad Sci USA 738:201-213.

Haïdara K, Moffatt P, Denizeau F (1999) Toxicol Sci 49:297-305.

Harris Z L, Takahashi Y, Miyajima H, Serizawa M, MacGillivray R T A, Gitlin J D (1995) Proc Natl Acad Sci USA 92:2539-2543.

Harris Z L, Durley A P, Man T K, Gitlin J D (1999) Proc Natl Acad Sci USA 96: 10812-10817.

Hartard C, Weisner B, Dieu C, Kunze K (1993) J Neurol 241:101-107.

Hartmann H A, Evenson M A (1992) Med Hypothesis 378:75-85.

Hatten M E (1999) Ann Rev Neurosci 22:511-539.

Hilton M, Spencer D C, Ross P, Ramsey A, McArdle H J (1995) Biochem Biophys Acta 1245:153-160.

Honing L S, Rosenberg M D (2000) Am J Med 108:317-330.

Jeannotte R, Paquin J, Petit-Turcofte C, Day R (1997) DNA Cell Biol 16:1175-1187.

Kataoka M, Tavassoli M (1985) Exp Hematol 13:806-810.

Katz B M, Barnea A (1990) J Biol Chem 265:2017-2021.

Klomp L W, Gitlin J D (1996) Hum Mol Genet 5:1989-1996.

Lindvall O, Kokaia Z, Bengzon J, Elmer E, Kokaia M (1994) Trends Neurosci 17:207-215.

Mateescu M, Chahine R, Roger S, Atanasiu R, Yamaguchi N, Lalumiere G, Nadeau R (1995) Arzneimittel-Forsch/Drug Res 45:476-480.

Mateescu M, Fortier G, Neidhardt S, Roger S (1988) Chromatographica 86:110-114.

Mateescu M A, Wang X T, Dumoulin M J, Befani O, Mondovi B (1999) in Analytical and preparative separation methods of biomacromolecules (Aboul-Enein H Y, ed), pp 445-455. New York: Marcel Dekker Inc.

McAuslan B R, Reily W G, Hannan G N, Gole G A (1983) Microvasc Res 26:323-338.

McBurney M W (1993) Int J Dev Biol 37:135-140.

Messerschmidt A, Huber R (1990) Eur J Biochem 187:341-352.

Mollgard K, Dziegielewska K M, Saunders N R, Zakut H, Soreq H (1988) Dev Biol 128:207-221.

Morell A G, Irvine R A, Sterlier I, Scheinberg I H (1968) J Biol Chem 243:155-159.

Mukhopadhyay C K, Attieh Z H, Fox P L (1998) Science 279:714-717.

Muller Y L, Yool A J (1998) Brain Res Dev Brain Res 108:193-203.

Mummery C L, van den Brink C E, de Laat S W (1987) Dev Biol 121:10-19.

Orena S J, Goode C A, Linder M C (1986) Biochem Biophys Res Commun 139:822-829.

Paquin J, Mateescu M A, De Grandpré E (1999) Patent pending CA00/00529 or WO 00/67782.

Parnas D, Linial M (1997) Dev Brain Res 101:115-124.

Patel B N, David S (1997) J Biol Chem 272:20185-20190.

Pemberton S, Lindley P, Zaitsev V, Card G, Tuddenham EGD, Kemball-Cook G (1997) Blood 89:2413-2421.

Puchkova L V, Verbina I A, Gaitskoki V S, Neifakh S A (1991) Biokhimiia 56:2261-2269 (English translation of abstract).

Puchkova L V, Sasina L K, Aleinikova T D, Zakharova E T, Gaitskhoki V S (1997) Biochemistry (Moscow) 62:697-703.

Salzer J L, Lovejoy L, Linder M C, Rosen C (1998) J Neurosci Res 54:147-157.

Sarkar B, Lingertat-Walsh K, Clarke J T R (1993) J Pediatr 123:828-830.

Seeds N W, Basham M E, Haffke S P (1999) Proc Natl Acad Sci USA 96:14118-14123.

Shea T B, Beermann M L (1992) FEBS Lett 307:190-194.

Stevens M O, Di Silvestro R A, Harris E D (1984) Biochemistry 23:261-266.

Thewke D P, Seeds N W (1999) Brain Res 821:356-367.

Waggoner D J, Bartnikas T B, Gitlin J D (1999) Neurobiol Dis 6:221-230.

Wang R, Zhang L, Mateescu M A, Nadeau R (1995) Biochem Biophys Res Commun 207:599-605.

Wu L, de Champlain J (1996) J Hypertension 14:593-599.

Wu L, Mateescu M A, Wang X T, Mondovi B, Wang R (1996) Biochem Biophys Res Commun 220:47-52.

Yoshida S, Shiosaka S (1999) Int J Mol Med 3: 405409.

Zaitseva I, Zaitsev V, Card G, Moshkov K, Bax B, Ralph A, Lindley P (1996) J Biol Inorg Chem 1:15-23.

While several embodiments of the invention have been described, it will be understood that the present invention is capable of further modifications, and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and falling within the scope of the invention or the limits of the appended claims.

1. A method for promoting aggregation of neuronal cells, characterized in that it comprises the step of providing to said neuronal cells an effective amount of ceruloplasmin and/or of a functional derivative thereof.

2. The method of claim 1, characterized in that said aggregation promotes tissular organization of said neuronal cells.

3. The method of claim 1 or 2, characterized in-that said neuronal cells consist of newly differentiated neurons or of highly developed neurons susceptible to undergo or to respond to a regenerative process.

4. The method of any one of claims 1 to 3, characterized in that said neuronal cells consist of in vitro cultured neuronal cells.

5. The method of claim 4, characterized in that said in vitro cultured neuronal cells are cultured for a subsequent transplantation in a mammal.

6. The method of any one of claims 1 to 3, characterized in that said neuronal cells are selected from the group consisting of neuronal cells of the brain neuronal cells of the spinal cord and neuronal cells of the peripheral nervous system of a living mammal.

7. The method of any one of claims 1 to 6, characterized in that ceruloplasmin and/or its functional derivative are purified from blood or produced by recombinant techniques.

8. The method of claim 7, characterized in that ceruloplasmin and/or its functional derivative are purified using a one-step affinity chromatography method and an affinity column comprising aminoethyl-agarose.

9. A method for promoting aggregation and/or for promoting tissular organization of neuronal cells in a living mammal, the method being characterized in that it comprises the step of administering to said mammal an effective amount of ceruloplasmin and/or of a functional derivative thereof.

10. The method of claim 9, characterized in that said neuronal cells consist of newly differentiated neurons or of highly developed neurons susceptible to undergo or to respond to a regenerative process.

11. The method of claim 9 or 10, characterized in that said neuronal cells are selected from the group consisting of neuronal cells of the brain, neuronal cells of the spinal cord and neuronal cells of the peripheral nervous system.

12. The method of any one of claims 9 to 11, characterized in that said living mammal consists of a human suffering or likely to suffer of a neurodegenerative disease.

13. The method of claim 12, characterized in that said neurodegenerative disease is selected from the group consisting of Alzheimers disease, trauma to tissues of the nervous system, multiple sclerosis, Parkinson's disease, Hallervoden-Spatz' disease, siderosis, HIV infection of the nervous system, AIDS dementia, amyotrophic lateral sclerosis, hereditary hemorrhage with amyloidosis Dutch type, cerebral amyloid angiopathy, cerebral amyloid angiopathy, Down's syndrome, spongiform encephalopathy, Creutzfeld-Jakob, Rett's syndrome, epilepsy, neuronal migration diseases, metabolic status or diseases affecting neuronal function/survival and nervous system damages caused by cessation of blood flow.

14. The method of any one of claims 9 to 13, characterized in that ceruloplasmin and/or its functional derivative are associated with a therapeutic agent.

15. The method of claim 14, characterized in that said therapeutic agent is selected from the group consisting of neurotransmitters, neuropeptides, hormones, trophic factors, neurotherapeutic chemical compounds, modulators of cell adhesion/migration, modulators of axonal sprouting, modulators of axonal guidance, modulators of synaptic connections and analogs thereof capable of by binding to tissues of the nervous system.

16. A method for promoting maturation, growth, tissular organization, regeneration and/or aggregation of neuronal cells cultured in vitro, comprising the step of providing to said in vitro cultured neuronal cells an effective amount of ceruloplasmin and/or of a functional derivative thereof.

17. Use of ceruloplasmin or of a functional derivative thereof as an active agent in the preparation of a medication for promoting aggregation and/or for promoting tissular organization of human neuronal cells.

18. A method for delivering specifically a therapeutic agent to neuronal cells, characterized in that it comprises the use of ceruloplasmin and/or of a functional derivative thereof as a carrier for said therapeutic agent.

19. The method of claim 18, characterized in that said therapeutic agent is delivered at the surface of the neuronal cells.

20. The method of claim 18 or 19, characterized in that ceruloplasmin or its functional derivative binds to a ceruloplasmin receptor that is present at the surface of the neuronal cells.

21. The method of any one of claims 18 to 20, characterized In that said therapeutic agent is coupled to ceruloplasmin or to the functional derivative of ceruloplasmin.

22. The method of any one of claims 18 to 21, characterized in that said neuronal cells are selected from the group consisting of neuronal cells of the brain, neuronal cells of the spinal cord and neuronal cells of the peripheral nervous system.

23. The method of any one of claims 18 to 22, characterized in that said therapeutic agent is selected from the group consisting of neurotransmitters, neuropeptides, hormones, trophic factors, neurotherapeutic chemical compounds, modulators of cell adhesion/migration, modulators of axonal sprouting, modulators of axonal guidance, modulators of synaptic connections and analogs thereof capable of by binding to tissues of the nervous system.

24. The method of any one of claims 1 to 23, characterized in that ceruloplasmin and/or its derivatives are coupled to a biocompatible polymer.

25. Use of ceruloplasmin or of a functional derivative thereof in as a carrier for delivering specifically a therapeutic agent to neuronal cells.

26. Use of ceruloplasmin or of a functional derivative thereof In a neurotrophic composition.

27. The use of claim 25 for protecting neuronal cells and/or for promoting maturation, growth, tissular organization, regeneration and/or aggregation of neuronal cells.

28. A pharmaceutical neurotrophic composition comprising:
ceruloplasmin and/or a functional derivative of ceruloplasmin in an amount effective to promote maturation, growth, tissular organization, regeneration and/or aggregation of neuronal cells; and
a suitable pharmaceutical acceptable diluent or carrier.

29. The neurotrophic composition of claim 27, wherein said neuronal cells consist of newly differentiated neurons or highly developed neurons susceptible to undergo or to respond to a regenerative process.

30. The neurotrophic composition of claim 27 or 28, wherein ceruloplasmin and/or its functional derivative are present in an amount varying from about 0.001 μM to about 20 μM.

31. The neurotrophic composition of claim 29, wherein ceruloplasmin and/or its functional derivative are present In an amount varying from about 0.01 μM to about 5 μM.

32. The neurotrophic composition of any one of claims 27 to 30, wherein ceruloplasmin and/or its functional derivative are purified from blood or produced by recombinant techniques.

33. The neurotrophic composition of claim 31, wherein ceruloplasmin and/or its functional derivative are purified using a one-step affinity chromatography method and an affinity column comprising aminoethyl-agarose.

34. The neurotrophic composition of any one of claims 27 to 32, further comprising an agent selected from the group consisting of metal chelators, metal scavengers, preserving agents, solubilizing agents, stabilizing agents, wetting agents, emulsifiers, sweeteners, colorants, odorants, salts, buffers, coating agents and antioxidants.

35. The neurotrophic composition of any one of claims 27 to 33, wherein ceruloplasmin and/or its functional derivative are associated with a therapeutic agent.

36. The neurotrophic composition of claim 34, wherein said therapeutic agent is selected from the group consisting of neurotransmitters, neuropeptides, hormones, trophic factors, neurotherapeautic chemical compounds, modulators of cell adhesion/migration, modulators of axonal sprouting, modulators of axonal guidance, modulators of synaptic connections and analogs thereof.

37. The neurotrophic composition of any one of claims 27 to 35 wherein ceruloplasmin and/or its derivatives are coupled to a biocompatible polymer.

38. A pharmaceutical neuron-delivery composition comprising:
ceruloplasmin and/or a functional derivative of ceruloplasmin as a carrier for delivering specifically a therapeutic agent to neuronal cells;
a therapeutic agent coupled to ceruloplasmin and/or said functional derivative;
a suitable pharmaceutical acceptable diluent or carrier.
